(19) United States
(12) Patent Application Publication
(10) Pub. No.: US 2013/0204104 A1
(43) Pub. Date: Aug. 8, 2013

(54) APPARATUS FOR EVALUATING A PATIENT'S HEMODYNAMIC STATUS USING A HEART-LUNG INTERACTION

(71) Applicants: Frederic Michard, Bievres (FR); Reinhold Knoll, Neuburg a.Inn, DE (US); Ulrich Pfeiffer, Munchen, DE (US)

(72) Inventors: Frederic Michard, Bievres (FR); Reinhold Knoll, Neuburg a.Inn, DE (US); Ulrich Pfeiffer, Munchen, DE (US)

(73) Assignee: Edwards Lifesciences IPRM AG, Nyon (CH)

(21) Appl. No.: 13/558,254

(22) Filed: Jul. 25, 2012

Related U.S. Application Data
(63) Continuation of application No. 11/700,500, filed on Jan. 31, 2007, now abandoned.

(30) Foreign Application Priority Data
Jan. 31, 2006 (DE) DE 102006004415

(51) Int. Cl. A61B 5/0205; A61M 16/00; A61B 5/0452; A61B 5/02; A61B 5/1455

(52) U.S. Cl. CPC A61B 5/0205; A61B 5/02028; A61B 5/14551; A61B 5/0452; A61M 16/0057
USPC 600/323; 600/484

(57) ABSTRACT

Apparatus for evaluating a mechanically ventilated patient's hemodynamic status, adapted to provide a respiratory variation diagram of a hemodynamic variable, and being capable of deriving the value of a hemodynamic parameter for each mechanical breath cycle as well as an assessment of its suitability for the hemodynamic analysis on basis of the respiratory variation diagram. A method is also provided.

This application is continuation of U.S. patent application Ser. No. 11/700,500, filed Jan. 31, 2007, priority is claimed to German patent application DE 10 2006004415.0, filed Jan. 31, 2006, the entire disclosure of which is hereby incorporated by reference herein.

The invention relates to an apparatus for evaluating a patient's hemodynamic status using heart-lung interaction induced hemodynamic analysis.

### BACKGROUND

It is generally known that for the healthcare management of patients undergoing surgery or who are critically ill clinical strategies are applicable. In particular, patients submitted to, for example, mechanical ventilation require reliable monitoring of their state of health for diagnostics or for deducing therapeutic measures.

It is an important goal of the healthcare management to maintain or improve perfusion of organs. Therefore, it is frequently appropriate to increase the patient's cardiac output (CO) using fluid therapy. The beneficial effect of fluid therapy is observed in approximately 50% of the patients, in the rest fluid therapy may be contraindicated because CO is either sufficient or, in case it is too low, it should primarily be increased with positive inotropic or vasoactive substances only.

For guiding fluid therapy the usage of the respiratory variation in hemodynamic variables is conventional, e.g. in arterial pressure, left ventricular stroke volume, pulse-oximetric plethysmographic waveform and pre-ejection period induced by mechanical ventilation. It is generally known that patients with significant respiratory variations in any of the above mentioned hemodynamic variables during mechanical ventilation are able to significantly improve their cardiac output (CO) in response to fluid therapy. Therefore, in order to identify whether a patient is able to benefit from the fluid therapy the patient's respiratory variations are observed.

For assessing the patient's respiratory variations a contour analysis thereof is performed using hemodynamic parameters. Known hemodynamic parameters are, e.g., pulse pressure variation and stroke volume variation, but also pulse-oximetric plethysmographic waveform variations and pre-ejection period variations

These parameters are appropriate for predicting the volume responsiveness and CO response.

From U.S. Pat. No. 6,585,658 and U.S. Pat. No. 5,769,082 methods are known for assessing the respiratory variation in arterial pressure during mechanical ventilation. By using these methods the effects of mechanical ventilation on arterial systolic pressure can be quantified. However, for performing these methods, a manipulation of ventilatory settings is required, either to induce apnea according to U.S. Pat. No. 6,585,658 or to apply incremental and standardized levels of the airway pressure according to U.S. Pat. No. 5,769,082.

### SUMMARY OF THE INVENTION

An object of the invention is to provide an apparatus for evaluating e.g. a mechanically ventilated patient's hemodynamic status, wherein the evaluation result is accurate, reliable and simple to achieve.

The present invention provides an apparatus for evaluating a mechanically ventilated patient's hemodynamic status, adapted to provide a respiratory variation diagram of a hemodynamic variable, and being capable of deriving the value of a hemodynamic parameter for each mechanical breath cycle as well as an assessment of its suitability for the hemodynamic analysis on basis of the respiratory variation diagram.

Due to the fact that according to the invention for each value of the hemodynamic parameter an assessment of its suitability for the hemodynamic analysis is performed, an identified non-suitable value of the hemodynamic parameter is not used for the hemodynamic analysis. Therefore, the results derived from the hemodynamic analysis are generated with suitable values of the hemodynamic parameter only. Hence, the results derived from the hemodynamic analysis are accurate and reliable.

Non-suitable values of the hemodynamic parameter occur in specific clinical situations, during which the quantification of the effects of mechanical ventilation on hemodynamic variables is not clinically relevant or even dangerous. These situations are, e.g., when the patient suffers from cardiac arrhythmia, or when the patient has irregular breathing patterns, i.e. an irregular respiratory frequency or an irregular tidal volume.

Further, since the hemodynamic parameter as well as the suitability assessment thereof is derived from the respiratory variation diagram, a manipulation of ventilatory settings is not required. Therefore, with the inventive apparatus the evaluation result is simple to achieve.

Preferably the hemodynamic variable is the arterial pulse pressure PP (the difference between the systolic and the preceding diastolic pressure) and the hemodynamic parameter is the arterial pulse pressure variation PPV.

Using the arterial pulse pressure PP for quantifying the effects of mechanical ventilation is informative to guide fluid therapy. Therefore, the evaluation result achieved using the inventive apparatus is accurate and reliable.

Further, according to a preferred embodiment of the invention, the apparatus includes any arterial catheter for measuring the arterial pulse pressure (PP).

It is preferred that the apparatus is adapted to make use of the equation

$$PPV = 2 \frac{PP max - PP min}{PP max + PP min} ,$$

wherein PP max is the maximum arterial pulse pressure PP per mechanical breath cycle, and PP min is the minimum arterial pulse pressure PP per mechanical breath cycle (PPV is often expressed as a percentage, therefore 200 may replace 2 in the above equation if a percentage expression is wanted).

According to an alternative preferred embodiment of the invention, the hemodynamic variable is the arterial systolic pressure and the hemodynamic parameter is the arterial systolic pressure variation, wherein the apparatus preferably includes an arterial catheter for measuring the arterial systolic pressure.

According to another alternative preferred embodiment of the invention, the hemodynamic variable is the left ventricular stroke volume and the hemodynamic parameter is the left ventricular stroke volume variation, wherein the apparatus preferably includes or is connected to equipment, such as a sensor, allowing the beat by beat measurement of the left ventricular stroke volume.

According to a further alternative preferred embodiment of the invention, the hemodynamic variable is the pulse oximetry plethysmographic waveform and the hemodynamic parameter is the pulse oximetry plethysmographic waveform variation, wherein the apparatus preferably includes or is connected to a pulse oximeter probe for measuring the pulse oximetry plethysmographic waveform.

According to another alternative preferred embodiment of the invention, the hemodynamic variable is the pre-ejection period and the hemodynamic parameter is the pre-ejection period variation, wherein the apparatus preferably includes means for simultaneously recording the ECG and either a pulse oximeter plethysmographic signal or an arterial pressure signal for determining the pre-ejection period, such as a recorder. The pre-ejection period is defined by Bendjelid, J Appl Physiol (2004) 96:337-342.

It is preferred that the apparatus is adapted to perform for each value of the hemodynamic parameter the assessment of the suitability thereof on basis of the detection of arrhythmia of the patient.

The apparatus is preferably adapted to detect arrhythmia of the patient by registering the time intervals between the beat-to-beat peaks of the hemodynamic variable, determining a mean time interval value on basis of the respiratory variation diagram, and detecting a mechanical breath cycle comprising at least one time interval exceeding a predetermined deviation from the mean time interval value in order to exclude the value of the hemodynamic parameter assigned to said mechanical breath cycle from the hemodynamic analysis.

The preferred predetermined deviation is 15% of the mean time interval value.

As an alternative, the apparatus is preferably adapted to detect arrhythmia of the patient by making use of an ECG.

Alternatively, it is preferred that the apparatus is adapted to register time intervals t between the beat-to-beat peaks of the arterial pulse pressure PP, determine a mean time interval value t̅ on basis of the respiratory variation diagram, and wherein the hemodynamic variable is the normalized pulse pressure PPn defined as

$$PPn = PP \frac{t}{\overline{t}} ,$$

and the hemodynamic parameter is the arterial pulse pressure variation PPV.

The method is further refined using the normalized pulse pressure PPn for calculating the arterial pulse pressure variation PPV, since values of the arterial pulse pressure variation PPV are even appropriate for hemodynamic analysis, when are extra systolic beats or other irregular heart beat patterns occur

Preferably the apparatus includes any arterial catheter for measuring the arterial pulse pressure (PP).

According to a preferred embodiment of the invention the apparatus is adapted to make use of the equation

$$PPV = 2 \frac{PPn max - PPn min}{PPn max + PPn min} ,$$

wherein PPn max is the maximum normalized arterial pulse pressure (PPn) per mechanical breath cycle, and PPn min is the minimum normalized arterial pulse pressure (PPn) per mechanical breath cycle (30). Because mean time interval for normalization is the same within in this formula. Mean time interval cancels out and could be replaced here by a constant e.g. 1.

Further, it is preferred that the apparatus is adapted to perform for each value of the hemodynamic parameter the assessment of the suitability thereof on basis of the detection of irregular breathing patterns of the patient.

Preferably the apparatus is adapted to detect irregular breathing patterns of the patient by registering the values of the hemodynamic parameter, and detecting at least one mechanical breath cycle pattern comprised of at least three consecutive mechanical breath cycles comprising the values of the hemodynamic parameter exceeding a predetermined deviation from each other in order to exclude the values of the hemodynamic parameter assigned to said mechanical breath cycle pattern from the hemodynamic analysis.

The preferred predetermined deviation is 15% of the mean value of the values of the hemodynamic parameter assigned to said mechanical breath cycle pattern.

As alternatives, it is preferred that the apparatus is adapted to detect irregular breathing patterns of the patient by making use of an airway pressure curve or an airway flow curve, or a central venous pressure curve or a capnographic curve.

As alternatives, it is preferred that the apparatus is adapted to detect irregular breathing patterns by tracking changes of chest dimensions in using either a thoracic bioimpedance signalor a respiratory inductive plethysmographic signal or a magnetometer system signal.

According to a preferred embodiment of the invention, the apparatus is adapted to display the respiratory variation diagram of the hemodynamic variable in such manner that the respiratory variation diagram is shown as vertical bar graph, wherein for each beat-to-beat hemodynamic variable an individual bar is plotted, which e.g. in case of pulse pressure could be defined between the diastolic and systolic pressure value for each beat.

### BRIEF DESCRIPTION OF THE DRAWINGS

In the following the invention is explained on the basis of a preferred embodiment with reference to the drawings. In the drawings:

FIG. 1 shows an embodiment of an apparatus according to the invention,

FIG. 2 shows four respiratory variation diagrams according to the invention,

FIG. 3 shows eight respiratory variation diagrams according to the invention,

FIG. 4 shows four respiratory variation diagrams, two of them including arrhythmia indications according to the invention,

FIG. 5 shows four respiratory variation diagrams including irregular breathing pattern indications according to the invention, and

FIG. 6 shows an alternative embodiment of an apparatus according to the invention.

### DETAILED DESCRIPTION

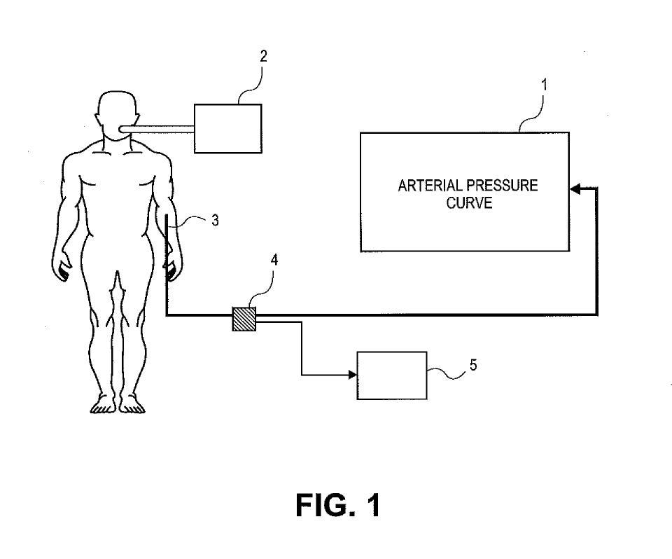

FIG. 1 shows a patient under mechanical ventilation, wherein the patient is ventilated by a ventilator 2 and instrumented with a basic configuration.

The basic configuration includes an arterial pressure transducer 4 connected via a catheter to an arterial line 3 of the patient. The arterial pressure transducer 4 sends measurement signals to a bedside monitor 5 as well as to an apparatus 1 according to the invention. The signals represent the arterial pulse pressure PP measured in line 3.

The apparatus 1 continuously receives the arterial pulse pressure PP signals from the arterial pressure transducer 4, generates a respiratory variation diagram on basis of the arterial pulse pressure PP signals, records and analyzes the respiratory variation diagrams continuously for performing a hemodynamic analysis.

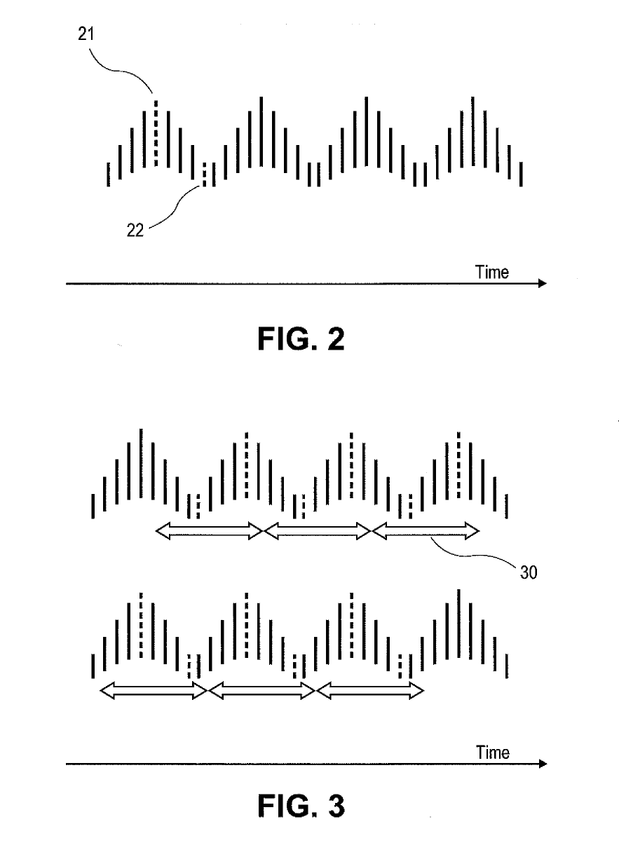

FIG. 2 shows four respiratory variation diagrams on basis of the arterial pulse pressure PP signals sent by the arterial pressure transducer 4 to the apparatus 1.

The respiratory variation diagram of the arterial pulse pressure PP is shown as vertical bar graph, wherein for each beat-to-beat curve section of the arterial pulse pressure PP an individual bar is plotted. Each bar represents the arterial pulse pressure PP which varies during each mechanical breath between a maximum value PP max 21 and a minimum value PP min 22.

FIG. 3 shows eight respiratory variation diagrams and arrows 30 indicating a single respiratory cycle.

The duration of each respiratory cycle is equal to 60/RF, where RF is the respiratory frequency expressed in 1/min,

The arterial pulse pressure variation PPV is calculated over successive respiratory cycles based on the respiratory variation diagram of the arterial pulse pressure PP by making use of the equation

$$PPV = 2 \frac{PP max - PP min}{PP max + PP min} .$$

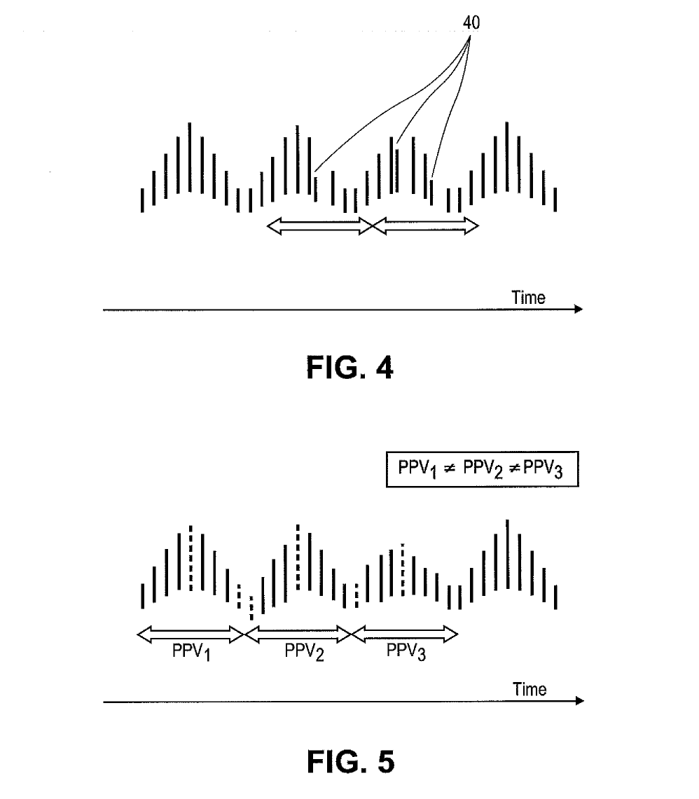

FIG. 4 shows a respiratory variation diagram of the arterial pulse pressure PP including arrhythmia indications 40.

The detection of arrhythmia is performed by an analysis of the tracing of the arterial pulse pressure PP in the respiratory variation diagram. In other words, the time intervals between all peaks (or bars) included in a respiratory cycle are measured. If the variability (defined as standard deviation divided by a mean time interval) of these time intervals is greater than a predetermined threshold value (e.g. 15%), this respiratory cycle is excluded from further hemodynamic analysis.

FIG. 5 shows four respiratory variation diagrams of the arterial pulse pressure PP in case of irregular breathing pattern (caused by an irregular tidal volume). PPmax and PPmin vary from one respiratory variation diagram to the other, so does PPV.

The detection of arrhythmia is performed by an analysis of the tracing of the arterial pulse pressure PP in the respiratory variation diagram, as illustrated in FIG. 4. The arterial pulse pressure variation PPV is calculated for each respiratory cycle without arrhythmia. If the variability (defined as standard deviation divided by a mean value of the arterial pulse pressure variation PPV) of at least three consecutive PPV values is greater than a predetermined threshold value (e.g. 15%), the corresponding PPV values will be considered as being invalid and are excluded from the hemodynamic analysis.

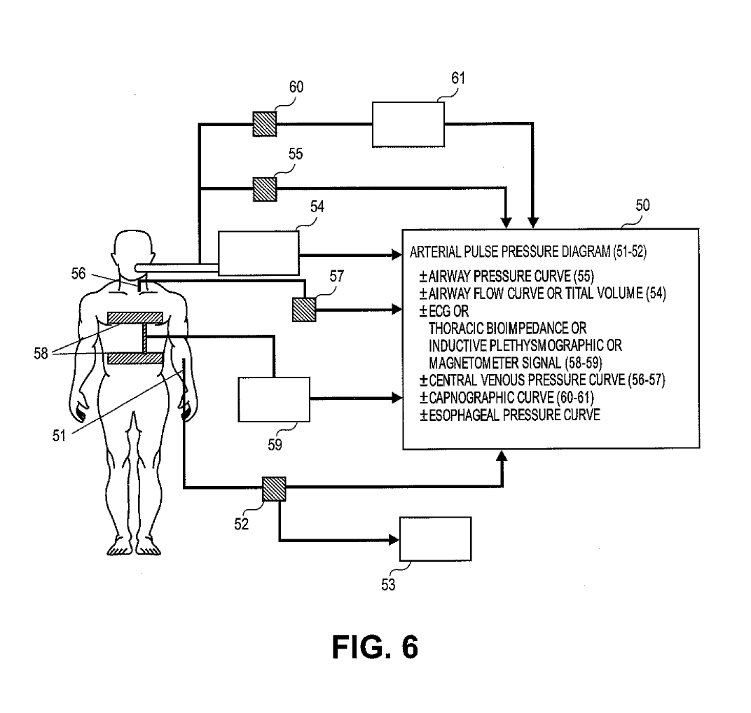

FIG. 6 shows a patient under mechanical ventilation, wherein the patient is ventilated by a ventilator 54 and instrumented with an alternative configuration.

The alternative configuration includes an arterial pressure transducer 52 connected via a catheter to an arterial line 51 of the patient. The arterial pressure transducer 52 sends measurement signals to a regular, standard bedside monitor 53 as well as to an apparatus 50 according to the invention. The signals represent the arterial pulse pressure PP measured in line 51.

Further, the alternative configuration includes a central venous pressure transducer 57 connected via a catheter to a central venous line 56 of the patient. The central venous pressure transducer 57 sends measurement signals to the apparatus 50.

Additionally, the alternative configuration includes an airway pressure transducer 55 connected via the respiratory circuit to the patient. The airway pressure transducer sends measurement signals to the apparatus 50.

Furthermore, the alternative configuration includes a ECG monitor or a thoracic bioimpedance monitor or a respiratory inductive plethysmography monitor or a magnetometer monitor 59 connected via electrodes (for ECG and thoracic bioimpedance) or elastic bands (for inductive plethysmography) or magnetometer coils (for the magnetometer system) 58 to the patient. The ECG monitor or the thoracic bioimpedance monitor or the respiratory inductive plethysmography monitor or the magnetometer monitor 59 sends measurement signals to the apparatus 50.

For CAPNOGRAPHIC MEASUREMENTS: additionally, the alternative configuration includes a CO2 sensor (60) on the respiratory circuit connected to a CO2 monitor (61). The CO2 monitor sends CO2 measurement signals to the apparatus.

In case of DIRECT CONNECTION WITH THE VENTILATOR: Additionally, the alternative configuration includes a connection between the ventilator and the apparatus. The ventilator sends tidal volume, or/and airway pressure, or/and airway flow measurement signals to the apparatus.

In case of SV measurement by ESOPHAGEAL or TRANSCUTANEOUS DOPPLER: Additionally, the alternative configuration includes an esophageal or transcutaneous Doppler probe connected to a Doppler monitor. The Doppler monitor sends stroke volume measurement signals to the apparatus.

The apparatus 50 continuously receives the signals from the arterial pressure transducer 52, the central venous pressure transducer 57, the airway pressure transducer 55 and the ECG monitor or thoracic bioimpedance monitor or respiratory inductive plethysmography monitor or magnetometer monitor 59 and the ventilator 54, and the CO2 monitor 61. On basis of these signals the apparatus 50 generates respectively an arterial pressure curve, a CVP curve, an airway pressure curve, an ECG tracing ora bioimpedance signal or a plethysmographic signal or a magnetometer signal, an airway flow and a tidal volume signals, and a capnographic signal.

The ECG is used for the detection of arrhythmia according to predefined algorithms; the airway pressure curve or the airway flow curve or the capnographic curve or the central venous pressure curve is used for the automatic detection of respiratory frequency and of irregular breathing pattern, e.g. caused by an irregular respiratory frequency or an irregular tidal volume; the thoracic bioimpedance signal or the respiratory inductive plethysmography signal or the magnetometer signal is used for the automatic detection of the respiratory frequency and irregular breathing patterns and the determination of tidal volume.

Taking the above into account, a process for evaluating a mechanically ventilated patient's hemodynamic status includes the steps:

Providing any arterial catheter to the patient and measuring the arterial pulse pressure PP with the artery catheter.

Providing a respiratory variation diagram of the arterial pulse pressure PP , the respiratory variation diagram comprising a vertical bar graph, wherein for each beat-to-beat curve section of the arterial pulse pressure PP an individual bar is provided,

Deriving the value of the arterial pulse pressure variation PPV for each mechanical breath cycle from the respiratory variation diagram of the arterial pulse pressure PP making use of the equation

$$PPV = 2 \frac{PP max - PP min}{PP max + PP min} ,$$

wherein PP max 21 is the maximum arterial pulse pressure PP per mechanical breath cycle 30, and PP min 22 is the minimum arterial pulse pressure PP per mechanical breath cycle 30.

Performing for each value of the arterial pulse pressure variation PPV an assessment of the suitability thereof on basis of the detection of arrhythmia of the patient by registering the time intervals between the beat-to-beat peaks of the hemodynamic variable, determining a mean time interval value on basis of the respiratory variation diagram, and detecting a mechanical breath cycle comprising at least one time interval exceeding a predetermined deviation from the mean time interval value, preferred are 15% of the mean time interval value, and eliminating the value of the arterial pulse pressure variation PPV assigned to said mechanical breath cycle.

Performing for each value of the hemodynamic parameter the assessment of the suitability thereof on basis of the detection of irregular breathing patterns of the patient by registering the values of the hemodynamic parameter, and detecting at least one mechanical breath cycle pattern comprised of at least three consecutive mechanical breath cycles comprising the values of the hemodynamic parameter exceeding a predetermined deviation from each other, preferred are 15% of the mean value of the values of the hemodynamic parameter assigned to said mechanical breath cycle pattern, and eliminating the values of the arterial pulse pressure variation PPV assigned to said mechanical breath cycle patterns.

Performing the hemodynamic analysis on basis of the non-eliminated values of the arterial pulse pressure variation PPV .

As an alternative to the arterial pulse pressure PP, the arterial pulse pressure variation PPV , the arterial systolic pressure, the arterial systolic pressure variation, or the left ventricular stroke volume, the left ventricular stroke volume variation and an equipment allowing the beat by beat measurement of the left ventricular stroke volume (e.g. arterial pulse contour analysis monitor or esophageal/transcutaneous Doppler monitor), or the pulse oximetry plethysmographic waveform, the pulse oximetry plethysmographic waveform variation and a pulse oximeter probe, or the pre-ejection period, the pre-ejection period variation and the ECG and either a pulse oximeter plethysmographic signal or an arterial pressure signal for determining the pre-ejection period can be used.

Alternatively an ECG can be used to detect arrhythmia of the patient.

As an alternative to the arterial pulse pressure PP and the arterial pulse pressure variation PPV , the normalized pulse pressure (PPn) can be used. The further steps have to be carried out, namely

Registering time intervals (t) between the beat-to-beat peaks of the arterial pulse pressure (PP), determining a mean time interval value ( t̅) on basis of the respiratory variation diagram, calculating the normalized pulse pressure (PPn) by making use of the equation

$$PPn = PP \frac{t}{\overline{t}} ,$$

and calculating the values of the arterial pulse pressure variation PPV making use of the equation

$$PPV = 2 \frac{PPn max - PPn min}{PPn max + PPn min} ,$$

wherein PPn max is the maximum normalized arterial pulse pressure PPn per mechanical breath cycle, and PPn min is the minimum normalized arterial pulse pressure PPn per mechanical breath cycle.

Alternatively an airway pressure curve or a central venous pressure curve or a thoracic bioimpedance signal or a tidal volume signal or an airway flow curve or a capnographic signal or a respiratory inductive plethysmographic signal or a magnetometer signal can be used to detect irregular breathing patterns of the patient.

The above mentioned process steps can be carried out by a computer program comprising appropriate instructions, and apparatus may be a controller such as a microprocessor or circuitry specifically designed for the present application, such as an ASIC.

What is claimed is:

1. An apparatus for evaluating a hemodynamic status of a mechanically ventilated patient, comprising:
a device adapted to analyze a respiratory variation of a hemodynamic variable, and being capable of deriving a value of a hemodynamic parameter for each mechanical breath cycle as well as an assessment of a suitability of the value of the hemodynamic parameter for a hemodynamic analysis on the basis of a respiratory variation diagram.

2. The apparatus according to claim 1, wherein the hemodynamic variable is an arterial pulse pressure (PP) and the hemodynamic parameter is an arterial pulse pressure variation (PPV).

3. The apparatus according to claim 2, further comprising an arterial catheter for measuring the arterial pulse pressure (PP).

4. The apparatus according to claim 2 wherein the apparatus is adapted to make use of the equation
$$PPV = 2 \frac{PP max - PP min}{PP max + PP min}$$
wherein PP max is a maximum arterial pulse pressure (PP) per mechanical breath cycle, and PP min is a minimum arterial pulse pressure (PP) per mechanical breath cycle.

5. The apparatus according to claim 1, wherein the hemodynamic variable is the arterial systolic pressure and the hemodynamic parameter is the arterial systolic pressure variation.

6. The apparatus according to claim 5, further comprising an arterial catheter for measuring the arterial systolic pressure.

7. The apparatus according to claim 1 wherein the hemodynamic variable is the left ventricular stroke volume and the hemodynamic parameter is the left ventricular stroke volume variation.

8. The apparatus according to claim 7, further comprising a sensor allowing the beat by beat measurement of the left ventricular stroke volume.

9. The apparatus according to claim 1 wherein the hemodynamic variable is the pulse oximetry plethysmographic waveform and the hemodynamic parameter is the pulse oximetry plethysmographic waveform variation.

10. The apparatus according to claim 9, further comprising a pulse oximeter probe for measuring the pulse oximetry plethysmographic waveform.

11. The apparatus according to claim 1 wherein the hemodynamie variable is the pre-ejection period and the hemodynamie parameter is the pre-ejection period variation.

12. The apparatus according to claim 11, further comprising a recorder for simultaneously recording the ECG and either a pulse oximeter plethysmographic signal or an arterial pressure signal for determining the pre-ejection period.

13. The apparatus according to claim 1 wherein the apparatus is adapted to perform for each value of the hemodynamic parameter the assessment of the suitability thereof on basis of the detection of arrhythmia of the patient.

14. The apparatus according to claim 13 wherein the apparatus is adapted to detect arrhythmia of the patient by registering time intervals between beat-to-beat peaks of the hemodynamic variable, determining a mean time interval value on basis of the respiratory variation diagram, and detecting a mechanical breath cycle comprising at least one time interval exceeding a predetermined deviation from the mean time interval value in order to exclude the value of the hemodynamic parameter assigned to said mechanical breath cycle from the hemodynamic analysis.

15. The apparatus according to claim 14 wherein the predetermined deviation is 15% of the mean time interval value.

16. The apparatus according to claim 13 wherein the apparatus is adapted to detect arrhythmia of the patient by making use of an ECG.

17. The apparatus according to claim 1 wherein the apparatus is adapted to register time intervals (t) between the beat-to-beat peaks of an arterial pulse pressure (PP), determine a mean time interval value ( t̅) on the basis of the respiratory variation diagram, and wherein the hemodynamic variable is the normalized pulse pressure (PPn) defined as
$$PPn = PP \frac{t}{\overline{t}} ,$$
and the hemodynamic parameter is the arterial pulse pressure variation (PPV).

18. The apparatus according to claim 17, further comprising an arterial catheter for measuring the arterial pulse pressure (PP).

19. The apparatus according to claim 17 wherein the apparatus is adapted to make use of the equation
$$PPV = 2 \frac{PPn max - PPn min}{PPn max + PPn min} ,$$
wherein PPn max is the maximum normalized arterial pulse pressure (PPn) per mechanical breath cycle, and PPn min is the minimum normalized arterial pulse pressure (PPn) per mechanical breath cycle (30).

20. The apparatus according to claim 1 wherein the apparatus is adapted to perform for each value of the hemodynamie parameter the assessment of the suitability thereof on the basis of the detection of irregular breathing patterns of the patient.

21. The apparatus according to claim 20 wherein the apparatus is adapted to detect irregular breathing patterns of the patient by registering the values of the hemodynamic parameter, and detecting at least one mechanical breath cycle pattern comprised of at least three consecutive mechanical breath cycles comprising the values of the hemodynamic parameter exceeding a predetermined deviation from each other in order to exclude the values of the hemodynamic parameter assigned to said mechanical breath cycle pattern from the hemodynamic analysis.

22. The apparatus according to claim 21 wherein the predetermined deviation is 15% of the mean value of the values of the hemodynamic parameter assigned to said mechanical breath cycle pattern.

23. The apparatus according to claim 20 wherein the apparatus is adapted to detect irregular breathing patterns of the patient by making use of an airway pressure curve or a central venous pressure curve or a thoracic bioimpedance signal or an airway flow curve, or a capnographic curve, or a respiratory inductive plethysmographic signal or a magnetometer signal or a tidal volume measurement.

24. The apparatus according to claim 1 wherein the apparatus is adapted to display the respiratory variation diagram of the hemodynamic variable in such manner that the respiratory variation diagram is shown as vertical bar graph, wherein for each beat-to-beat curve section of the hemodynamic variable an individual bar is plotted, which is defined between the maximum value and the minimum value of beat-to-beat curve section of the hemodynamic variable.

25. A method for evaluating a hemodynamic status of a mechanically ventilated patient, comprising:
analyzing a respiratory variation of a hemodynamic variable;
deriving the value of a hemodynamic parameter for each mechanical breath cycle; and
assessing of a suitability of the value of the hemodynamic parameter for a hemodynamic analysis on the basis of a respiratory variation diagram.
